(19) United States
(12) Patent Application Publication
(10) Pub. No.: US 2013/0303395 A1
(43) Pub. Date: Nov. 14, 2013

(54) MARKER SEQUENCES FOR SYSTEMIC LUPUS ERYTHEMATOSUS AND THE USE THEREOF

(71) Applicants: Angelika Lueking, Bochum (DE); Axel Kowald, Bochum (DE); Stefan Müllner, Langenfeld (DE); Christian Scheer, Dortmund (DE); Matthias Schneider, Dusseldorf (DE)

(72) Inventors: Angelika Lueking, Bochum (DE); Axel Kowald, Bochum (DE); Stefan Müllner, Langenfeld (DE); Christian Scheer, Dortmund (DE); Matthias Schneider, Dusseldorf (DE)

(73) Assignee: PROTAGEN AKTIENGESELLSCHAFT, DORTMUND (DE)

(21) Appl. No.: 13/879,149

(22) PCT Filed: Oct. 12, 2011
(86) PCT No.: PCT/EP11/67842

§ 371 Date: Jul. 31, 2013

(30) Foreign Application Priority Data
Oct. 12, 2010 (EP) 10187316.4

(51) Int. Cl. G01N 33/68

(52) U.S. Cl. CPC G01N 33/6854
USPC 506/9; 506/17

(57) ABSTRACT

The invention relates to novel marker sequences for systemic lupus erythematosus and to the use thereof in diagnosis as well as to a method for screening potential active ingredients for systemic lupus erythematosus diseases using said marker sequences. The invention further relates to a diagnostic device containing such marker sequences for systemic lupus erythematosus, especially to a protein biochip and the use thereof.

The present invention relates to novel marker sequences for systemic lupus erythematosus and to the use thereof in diagnosis, together with a method for screening potential active ingredients for systemic lupus erythematosus diseases by way of these marker sequences. The invention further relates to a diagnostic device comprising such marker sequences for systemic lupus erythematosus, in particular a protein biochip, and to the use thereof.

Protein biochips are gaining increasing industrial importance for analytical and diagnostic purposes as well as in pharmaceutical development. Protein biochips have also become established as screening tools.

To this end, the fast and highly parallel detection of a plurality of specifically binding analysis molecules during a single experiment is made possible. Producing protein biochips requires the availability of the necessary proteins. For this purpose, in particular protein expression libraries have become established. High throughput cloning of defined open reading frames is one option (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S, and Vidal, M. (2000) GATEWAY recombinational cloning: application to the cloning of large numbers of open reading frames or ORFeomes. Methods Enzymol, 328, 575-592). However, such an approach is highly dependent on the progress of genome sequencing projects and the annotation of these gene sequences. Moreover, the determination of the expressed sequence can be ambiguous due to differential splicing processes. This problem can be circumvented by the use of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr, Purif., 20, 372-378). To this end, the cDNA of a particular tissue is cloned into a bacterial or eukaryotic expression vector, such as yeast. The vectors used for expression are generally characterized in that these carry inducible promoters, by way of which the time of protein expression can be controlled. In addition, expression vectors comprise sequences for so-called affinity epitopes or affinity proteins, which permit the specific detection of recombinant fusion proteins by way of an antibody that is directed against the affinity epitope and additionally enable specific purification by way of affinity chromatography (IMAC).

For example, the gene products of a cDNA expression library from human fetal brain tissue in the bacterial expression system Escherichia coli were arranged in a high-density format on a membrane and able to be successfully screened with various antibodies. It was shown that the proportion of full-length proteins was at least 66%. It was further possible to express the recombinant proteins from expression libraries in high throughput and purify them (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Buessow (2000) supra; Lueking, A., Horn, M., Eickhoff, H., Buessow, K., Lehrach, H. and Walter, G. (1999) Protein microarrays for gene expression and antibody screening. Analytical Biochemistry, 270, 103-111). Such cDNA expression library-based protein biochips are the subject matter in particular of WO 99/57311 and WO 99/57312. In addition to antigen-presenting protein biochips, antibody-presenting arrangements are described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a high need to make indication-specific diagnostic devices, such as a protein biochip, available.

The object of the present invention is to provide improved marker sequences and the diagnostic use thereof for treating systemic lupus erythematosus.

The provision of specific marker sequences allows a reliable diagnosis and stratification of patients with systemic lupus erythematosus, in particular by way of a protein biochip.

The invention therefore relates to the use of marker sequences for diagnosing systemic lupus erythematosus, wherein at least one marker sequence of a cDNA selected from the group SEQ 1-716, or a respective protein coding therefor, or a respective partial sequence or fragment thereof (hereinafter: marker sequences according to the invention) is determined on or from a patient to be examined.

It was possible to identify the marker sequences according to the invention by way of differential screening of samples, specifically from healthy participants, with samples from patients with systemic lupus erythematosus.

For the first time, these marker sequences according to the invention were identified by way of protein chips (see examples).

The term “systemic lupus erythematosus (SLE)” relates a systemtic autoimmune disease from the collagenosis group. A particular characteristic of SLE (systemic lupus erythematosus) is the so-called butterfly rash.

Criteria for diagnosis are:

1. Butterfly rash
2. Discoid skin changes
3. Sensitivity to light
4. Mucous membrane ulcers (generally painless)
5. Arthritis in at least two joints
6. Serositis (pleurisy or pericarditis)
7. Kidney involvement (proteinuria <0.5 g/d or cylinder)
8. CNS involvement (seizures or psychosis)
9. Hematological findings (hemolytic anemia, leucopenia or thrombopenia)
10. Immunological findings: anti-dsDNA antibodies, anti-Sm antibodies, anticardiolipin antibodies;
11. Antinuclear antibodies without taking lupus erythematosus-triggering medication;

Evaluation: With four (three) positive findings, the diagnosis is considered reliable (likely) (definition e.g. according to Pschyrembel, de Gruyter, 261^{st} edition (2007), Berlin).

It is essential for the invention that the samples are not taken from conventional blood banks, but were carefully selected from SLE patients who are HIV and HCV negative, for example, and were tested in particular for infectious diseases. The complex sample selection procedure allows, for example, sufficient advantageous differentiation from diseases with symptoms similar to SME.False positive results are thus excluded, also because of the strict bioinformational evaluation (see examples).

The protein biochips are additionally produced by normalizing at least 1,000, preferably 2,000 different, or more, autoantigens of humans, which are not indication-specific of systemic lupus erythematosus. For example, such autoantigens can be obtained from other bodily fluids of patients with other illnesses such as pancreatic cancer, rheumatoid arthritis, prostate and the like.

The invention therefore also relates to such indication-specific protein biochips according to the invention for diagnosing systemic lupus erythematosus, wherein in a further verification step, the proteins represented on the protein biochip are normalized with autoantibodies from non-systemic lupus erythematosus patients and false positive proteins can be eliminated in this way.

Hereafter, a protein biochip thus produced is referred to as a “normalized protein biochip”. Such a normalized protein biochip has been found to be particularly advantageous for identifying specific marker sequences according to the invention because other similar autoimmune-relevant marker sequences can be excluded. This is contrary to the teaching of WO2003/090694.

This procedure likewise allows autoantibodies with a positive response to E. coli to be excluded. This is a further qualitative improvement, for example because autoantibodies that are directed to E. coli enterobacteria in humans can be excluded. As a result, new marker sequences can advantageously be identified with an improved signal-to-noise ratio.

In a further preferred embodiment, at least 2 to 5 or 10, preferably 30 to 50 marker sequences, or 50 to 100 or more marker sequences are thus determined on or from a patient to be examined.

In a further embodiment of the invention, the marker sequences according to the invention can also be combined, supplemented or expanded with known biomarkers for this indication.

In a preferred embodiment, the marker sequences are determined outside the human body and the determination is carried out in an ex vivo/in vitro diagnosis.

In a further embodiment of the invention, the invention relates to the use of marker sequences as diagnostic products, wherein at least one marker sequence of a cDNA is selected from the group SEQ 1-716, or a respective protein coding therefor, or a respective partial sequence or fragment thereof.

The invention further relates to a method for diagnosing systemic lupus erythematosus, wherein a.) at least one marker sequence of a cDNA selected from the group SEQ 1-716, or a protein coding therefor, or a respective partial sequence or fragment thereof, is applied to a solid support, and b.) brought in contact with body fluid or tissue extract and c.) the detection of an interaction of the body fluid or tissue extract with the marker sequences from a.) is carried out.

The invention therefore also relates to diagnostic products for diagnosing systemic lupus erythematosus, each selected from the group SEQ 1-716, or a respective protein coding therefor, or a respective partial sequence or fragment thereof.

Such an interaction can be detected by a probe, in particular by an antibody, for example.

The invention therefore also relates to the problem of providing a diagnostic device or an array, in particular a protein biochip, which allows a diagnosis of or examination for systemic lupus erythematosus.

The invention further relates to a method for the stratification, in particular for the risk stratification, and/or treatment management of a patient with systemic lupus erythematosus, wherein at least one marker sequence of a cDNA selected from the group SEQ 1-716, or a respective protein coding therefor, is determined on a patient to be examined.

The invention further encompasses the stratification of patients with systemic lupus erythematosus into new or established sub-groups of systemic lupus erythematosus as well as the expedient selection of patient groups for the clinical development of new therapeutic agents. The term treatment management also includes dividing the patients into responders and non-responders with respect to a treatment or the treatment course thereof.

The term “diagnosis” within the meaning of the present invention denotes the positive identification of systemic lupus erythematosus by way of the marker sequences according to the invention and the association of patients with the systemic lupus erythematosus disease. The term ‘diagnosis’ comprises medical diagnostics and examinations in this regard, in particular in vitro diagnostics and laboratory diagnostics, as well as proteomics and nucleic acid blotting. Additional examinations may be required for validation and to exclude other illnesses. The term ‘diagnosis’ therefore likewise encompasses the differential diagnosis of systemic lupus erythematosus by way of the marker sequences according to the invention and the prognosis of systemic lupus erythematosus.

“Stratifying (also: stratification) or treatment management” within the meaning of the present invention shall mean that the method according to the invention allows decisions regarding the treatment and therapy of the patient, be it hospitalization of the patent, use, effect and/or dosage of one or more pharmaceuticals, a therapeutic measure or monitoring the progression of an illness or treatment, or etiology or classification of a disease, for example into a new or existing sub-type, or the differentiation of illnesses and the patients thereof.

In a further embodiment of the invention, the term “stratification” comprises in particular risk stratification with the prognosis of an outcome of a disadvantageous health event.

Within the scope of the present invention, the term “patient” is considered to mean any participant—human or mammal—with the proviso that the participant is examined for systemic lupus erythematosus.

The term “marker sequences” within the meaning of the present invention shall mean that the cDNA, or the respective polypeptide or protein obtainable therefrom, is significant for systemic lupus erythematosus. For example, the cDNA, or the respective polypeptide or protein obtainable therefrom, can exhibit an interaction with substances from the body fluid or tissue extract of a patient with systemic lupus erythematosus (for example antigen (epitope)/antibody (paratope) interaction). Within the meaning of the invention, “wherein at least one marker sequence of a cDNA selected from the group SEQ 1-716, or a respective protein coding therefor, or a respective partial sequence or fragment thereof, is determined on or from a patient to be examined” shall mean that an interaction between the body fluid or tissue extract of a patient and the marker sequences according to the invention is detected. Such an interaction includes, for example, a bond, in particular a binding substance on at least one marker sequence according to the invention or, in the case of cDNA, hybridization with a suitable substance under select conditions, in particular stringent conditions (for example as customarily defined in J. Sambrook, E. F. Fritsch, T. Maniatis (1989), Molecular cloning: A laboratory manual, 2nd Edition, Cold Spring Habor Laboratory Press, Cold Spring Habor, USA or Ausubel, “Current Protocols in Molecular Biology”, Green Publishing Associates and Wiley Interscience, N.Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. One example of less stringent hybridization conditions is hybridization in 4s SCC at 37° C., followed by several washing steps in 1×SCC at room temperature.

According to the invention, such substances are constituents of a body fluid, in particular blood, whole blood, blood plasma, serum, patient serum, urine, cerebrospinal fluid, synovial fluid or a tissue extract of the patient.

In a further embodiment of the invention, however, the marker sequences according to the invention can be present in a significantly higher or lower expression rate or concentration, indicating systemic lupus erythematosus. To this end, the relative sick/healthy expression rates of the marker sequences for systemic lupus erythematosus according to the invention can be determined by way of proteomics or nucleic acid blotting.

In a further embodiment of the invention, the marker sequences comprise a detection signal that is addressed to the substance to be bound (for example antibody, nucleic acid). According to the invention, the detection signal is preferably an epitope and/or paratope and/or haptene for a protein, and it is a hybridization region or binding region for a cDNA.

The marker sequences according to the invention are likewise listed in Table A and can be unambiguously identified by the respective cited database entry (also via the Internet: http://www.ncbi.nlm.nih.gov/) (see in Table A: Accession No. there), see also the associated sequence protocol.

The invention thus likewise relates to full-length sequences of the markers according to the invention, more particularly as defined in Table 1 by way of the known database entry, hereafter referred to as SEQ 1a-716a (cDNA).

The invention therefore also comprises embodiments of SEQ 1a-716a that are analogous to the marker sequences SEQ 1-716, as described in the claims for example, because the SEQ 1-716 according to the invention again represent partial sequences, at least with high homology. However, the specific marker sequences SEQ 1-716 are preferred according to the invention.

According to the invention, the marker sequences also comprise modifications of the cDNA sequence, and of the corresponding amino acid sequence, such as chemical modification, for example citrullination, acetylation, phosphorylation, glycosylation or polyA tail and other relevant modifications known to a person skilled in the art.

Another embodiment of the invention also encompasses partial sequences or fragments of the marker sequences according to the invention. These are in particular partial sequences that are 95%, 90%, notably 80% or 70% identical to the marker sequences according to the invention.

Partial sequences also include sequences that comprise 50 to 100 nucleotides, 70 to 120 nucleotides of a sequence of SEQ 1-716, or peptides obtainable therefrom.

“Partial sequences or fragments” of the marker sequences according to the invention are functionally defined and comprise sequences that have the same diagnostic function according to the invention.

In a further embodiment, the respective marker sequence may be represented in differing quantities in one or more regions on a solid support. This allows the sensitivity to be varied. The regions can each comprise a collectivity of marker sequences, which is to say a sufficient number of different marker sequences, in particular 2 to 5, or 10 or more, and optionally additional nucleic acids and/or proteins, in particular biomarkers. However, at least 96 to 25,000 (numerical) or more different or identical marker sequences and additional nucleic acids and/or proteins, in particular biomarkers, are preferred. Further preferred are more than 2,500, particularly preferred are 10,000 or more different or identical marker sequences and optionally additional nucleic acids and/or proteins, in particular biomarkers.

Another object of the invention is an arrangement of marker sequences comprising at least one marker sequence of a cDNA selected from the group SEQ 1-716, or a respective protein coding therefor. The arrangement preferably comprises at least 2 to 5 or 10, preferably 30 to 50 marker sequences, or 50 to 100 or more marker sequences.

Within the scope of the present invention, “arrangement” shall be synonymous with “array”, and provided that this “array” is used to identify substances on marker sequences, this shall be understood to mean an “assay” or a diagnostic device. In a preferred embodiment, the arrangement is designed so that the marker sequences represented on the arrangement are present in the form of a grid on a solid support. Moreover, arrangements that allow a high-density arrangement of protein binders and where the marker sequences are spotted are preferred. Such high-density spotted arrangements are disclosed in WO 99/57311 and WO 99/57312, for example, and can advantageously be employed in a robot-assisted automated high-throughput method.

However, within the scope of the present invention the term “assay” or diagnostic device also comprises embodiments of a device, such as ELISA, bead-based assay, line assay, western blot, immunochromatographic methods (for example so-called lateral flow immunoassays) or similar immunological single or multiplex detection methods. A protein biochip within the meaning of the present invention is a systematic arrangement of proteins on a solid support.

The marker sequences of the arrangement are fixed on a solid support, however preferably they are spotted or immobilized, even printed on, which is to say they are applied reproducibly. One or more marker sequences can be present multiple times in the collectivity of all marker sequences and be present in differing quantities relative to a spot. Furthermore, the marker sequences can be standardized on the solid support (for example by way of human globulin serial dilution series as internal calibrators for data normalization and quantitative evaluation).

As a result, the invention also relates to an assay or a protein biochip comprising an arrangement containing marker sequences according to the invention.

In a further embodiment, the marker sequences are present in the form of clones. For example, such clones can be obtained by way of a cDNA expression library according to the invention (Büssow et al. 1998 (supra)). In a preferred embodiment, such expression libraries containing clones are obtained by way of expression vectors from an expressing cDNA library comprising the cDNA marker sequences. These expression vectors preferably comprise inducible promoters. The expression can, for example, be induced by way of an inducer such as IPTG. Suitable expression vectors are described in Terpe et al. (Terpe T Appl Microbiol Biotechnol. 2003 January; 60(5):523-33).

Expression libraries are known to a person skilled in the art and can be produced according to standard reference books such as Sambrook et al, “Molecular Cloning, A laboratory handbook, 2nd edition (1989), CSH press, Cold Spring Harbor, N.Y. Also preferred are expression libraries that are tissue-specific (for example human tissue, in particular human organs). According to the invention, expression libraries that can be obtained by way of exon trapping are also covered. The term ‘expression bank’ can be employed synonymously for the term expression library.

Also preferred are protein biochips or corresponding expression libraries that have no redundancy (so-called: Uniclone® library) and that can be produced according to the teachings of WO 99/57311 and WO 99/57312, for example. These preferred Uniclone libraries have a high proportion of non-defective fully expressed proteins of a cDNA expression library.

Within the scope of the present invention, the clones can also be, but are not limited to, transformed bacteria, recombinant phages or transformed cells from mammals, insects, fungi, yeast or plants.

The clones are fixed, spotted or immobilized on a solid support.

The invention thus relates to an arrangement, wherein the marker sequences are present in the form of clones.

The marker sequences can also be present in the form of a fusion protein comprising at least one affinity epitope or “tag”, for example. The tag may be one such as c-myc, his tag, arg tag, FLAG, alkaline phosphatase, V5 tag, T7 tag or strep tag, HAT tag, NusA, S tag, SBP tag, thioredoxin, DsbA, including a fusion protein, preferably a cellulose-binding domain, green fluorescent protein, maltose-binding protein, calmodulin-binding protein, glutathione S-transferase or lacZ.

In all embodiments, the term “solid support” encompasses designs such as a filter, a membrane, a magnetic or fluorophore-labeled bead, a silicon wafer, glass, metal, plastic material, a chip, a mass spectrometry target or a matrix. However, a filter is preferred according to the invention.

Moreover, PVDF, nitrocellulose or nylon are preferred filters (for example Immobilon P Millipore, Protran Whatman, Hybond N+Amersham).

In a further preferred embodiment of the arrangement according to the invention, this arrangement corresponds to a grid having the size of a microtiter plate (8-12 wells, 96 wells, 384 wells or more), a silicon wafer, a chip, a mass spectrometry target or a matrix.

In a further embodiment, the invention relates to an assay or protein biochip for identifying and characterizing a substance for systemic lupus erythematosus, characterized in that an arrangement or assay according to the invention a.) is brought in contact with at least one substance to be analyzed and b.) successful binding is detected.

The invention further relates to a method for identifying and characterizing a substance for systemic lupus erythematosus, characterized in that an arrangement or assay according to the invention a.) is brought in contact with at least one substance to be analyzed and b.) successful binding is detected.

The substance to be analyzed can be any arbitrary native or non-native biomolecule, a synthetic chemical molecule, a mixture, or a substance library.

After the substance to be analyzed has come in contact with a marker sequence, the successful binding process is evaluated, which takes place, for example, using commercially available image analysis software (GenePix Pro (Axon Laboratores), Aida (Raytest), ScanArray (Packard Bioscience)).

The protein-protein interactions according to the invention (for example protein on marker sequence, such as antigen/antibody) or corresponding “means for detecting successful binding” can be visualized, for example, in the customary manner by way of fluorescent labeling, biotinylation, radioisotope labeling or colloidal gold or latex particle labeling. Bound antibodies are detected with the aid of secondary antibodies labeled with commercially available reporter molecules (for example Cy, Alexa, Dyomics, FITC or similar fluorescent dyes, colloidal gold or latex particles), or with reporter enzymes, such as alkaline phosphatase, horseradish peroxidase, or the like, and the corresponding colorimetric, fluorescent or chemiluminescent substrates. Readout is carried out, for example, by way of a microarray laser scanner, a CCD camera or visually.

In a further embodiment, the invention relates to a pharmaceutical/active ingredient or prodrug developed for systemic lupus erythematosus and obtainable through the use of the assay or protein biochip according to the invention.

The invention therefore likewise relates to the use of an arrangement according to the invention or an assay for screening active ingredients for systemic lupus erythematosus.

In a further embodiment, the invention therefore likewise relates to a target for the treatment and therapy of systemic lupus erythematosus, selected in each case from the group SEQ 1-716 or a protein coding therefor.

In a further embodiment, the invention likewise relates to the use of the marker sequences according to the invention, preferably in the form of an arrangement as an affinity material for carrying out apheresis or, in the broader sense, dialysis, wherein substances from body fluids of a patient with systemic lupus erythematosus, such as blood or plasma, bind to the marker sequences according to the invention and consequently can be selectively withdrawn from the body fluid.

### EXAMPLES AND FIGURES

Ten or more patient samples were individually screened against a cDNA expression library. The systemic lupus erythematosus-specific expression clones were determined by way of comparison to ten or more healthy samples. The identity of the marker sequences was determined by way of DNA sequencing.

FIG. 1 shows the differential screening between two protein biochips from a cDNA expression library of a patient and a healthy participant, respectively. The differential clones are detected by way of fluorescent labeling and evaluated by way of bioinformatics.

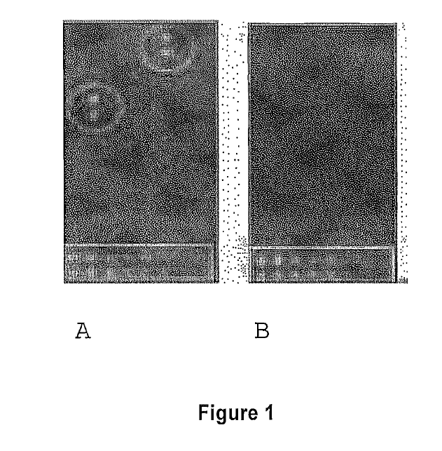

Within the scope of the biomarker identification, various bioinformatics analyses are carried out. Reactivities against approximately 2000 different antigens are measured for each serum using microarrays. This data is used to rank the spotted antigens with respect to the differentiation capability thereof between healthy and diseased sera. This evaluation is carried out by way of the non-parametric Mann-Whitney tests using normalized intensity data. An internal standard, which is also spotted on each chip, is used for normalization purposes. Because a p-value is calculated for each antigen, methods for correcting multiple testing are employed. A very conservative approach that is taken is to carry out a Bonferroni correction, and additionally the less restrictive false discovery rate (FDR) according to Benjamini & Hochberg is calculated.

Additionally, the data is utilized to classify the sera. To this end, different multivariate methods are employed. These are methods selected from statistical learning methods such as support vector machines (SVM), neuronal networks or classification trees, as well as a threshold value method, which is suitable both for classifying and for visually representing the data.

So as to avoid overfitting, tenfold cross validation of the data is carried out.

TABLE A
(gi accession number valid as of Oct. 1, 2010)

| SEQ 1a-716a | Name |
|---|---|
| gi\|66932947 | alpha-2-macroglobulin precursor [Homo sapiens] |
| gi\|55743075 | angio-associated, migratory cell protein [Homo sapiens] |
| gi\|45446740 | ATP-binding cassette, sub-family A, member 2 isoform a [Homo sapiens] |
| gi\|21536349 | amiloride-sensitive cation channel 2, neuronal isoform b [Homo sapiens] |
| gi\|4501867 | aconitase 2 precursor [Homo sapiens] |
| gi\|4501885 | beta actin [Homo sapiens] |
| gi\|4501887 | actin, gamma 1 propeptide [Homo sapiens] |
| gi\|29826323 | adducin 1 (alpha) isoform c [Homo sapiens] |
| gi\|194353970 | alpha-2A-adrenergic receptor [Homo sapiens] |
| gi\|148539876 | beta adrenergic receptor kinase 1 [Homo sapiens] |
| gi\|4501989 | alpha-fetoprotein precursor [Homo sapiens] |
| gi\|156523970 | alpha-2-HS-glycoprotein [Homo sapiens] |
| gi\|4502027 | albumin preproprotein [Homo sapiens] |
| gi\|4557305 | aldolase A [Homo sapiens] |
| gi\|32967601 | ankyrin 3 isoform 1 [Homo sapiens] |
| gi\|55749577 | solute carrier family 25 (mitochondrial carrier; adenine nucleotide translocator), |
| | member 4 [Homo sapiens] |
| gi\|156071459 | solute carrier family 25, member 5 [Homo sapiens] |
| gi\|156071462 | solute carrier family 25, member A6 [Homo sapiens] |
| gi\|4502131 | amyloid beta A4 precursor protein-binding, family B, member 1 isoform E9 [Homo |
| | sapiens] |
| gi\|4885065 | amyloid precursor-like protein 1 isoform 2 precursor [Homo sapiens] |
| gi\|4502201 | ADP-ribosylation factor 1 [Homo sapiens] |
| gi\|4502209 | ADP-ribosylation factor 5 [Homo sapiens] |
| gi\|33469974 | activating transcription factor 4 [Homo sapiens] |
| gi\|49574491 | Na+/K+-ATPase beta 2 subunit [Homo sapiens] |
| gi\|4757810 | ATP synthase, H+ transporting, mitochondrial F1 complex, alpha subunit precursor |
| | [Homo sapiens] |
| gi\|32189394 | ATP synthase, H+ transporting, mitochondrial F1 complex, beta subunit precursor |
| | [Homo sapiens] |
| gi\|50593533 | ATP synthase, H+ transporting, mitochondrial F0 complex, subunit C2 isoform a |
| | precursor [Homo sapiens] |
| gi\|4502313 | ATPase, H+ transporting, lysosomal, V0 subunit c [Homo sapiens] |
| gi\|20336205 | transcriptional regulator ATRX isoform 2 [Homo sapiens] |
| gi\|4502337 | alpha-2-glycoprotein 1, zinc [Homo sapiens] |
| gi\|115387099 | brain-specific angiogenesis inhibitor 2 [Homo sapiens] |
| gi\|178557739 | complement component 4B preproprotein [Homo sapiens] |
| gi\|8393009 | chromosome 11 open reading frame2 [Homo sapiens] |
| gi\|40804468 | calcium channel, voltage-dependent, beta 1 subunit isoform 1 [Homo sapiens] |
| gi\|4502549 | calmodulin 2 [Homo sapiens] |
| gi\|4502565 | calpain, small subunit 1 [Homo sapiens] |
| gi\|4502679 | CD63 antigen isoform A [Homo sapiens] |
| gi\|10835071 | CD74 antigen isoform b [Homo sapiens] |
| gi\|119627240 | cyclin-dependent kinase inhibitor 2C (p18, inhibits CDK4), isoform CRA_b [Homo |
| | sapiens] |
| gi\|56786147 | cysteine dioxygenase, type I [Homo sapiens] |
| gi\|169636439 | cholesteryl ester transfer protein, plasma precursor [Homo sapiens] |
| gi\|5031635 | cofilin 1 (non-muscle) [Homo sapiens] |
| gi\|4502805 | chromogranin A precursor [Homo sapiens] |
| gi\|6978649 | choline kinase beta isoform a [Homo sapiens] |
| gi\|21536286 | brain creatine kinase [Homo sapiens] |
| gi\|4502847 | cold inducible RNA binding protein [Homo sapiens] |
| gi\|14917109 | adaptor-related protein complex 2, mu 1 subunit isoform a [Homo sapiens] |
| gi\|4557471 | adaptor-related protein complex 1, sigma 1 subunit [Homo sapiens] |
| gi\|41872374 | polo-like kinase 3 [Homo sapiens] |
| gi\|4502981 | cytochrome c oxidase subunit IV isoform 1 precursor [Homo sapiens] |
| gi\|4503049 | cysteine-rich protein 2 [Homo sapiens] |
| gi\|4503051 | collapsin response mediator protein 1 isoform 2 [Homo sapiens] |
| gi\|4503065 | crystallin, mu isoform 1 [Homo sapiens] |
| gi\|4503101 | cysteine and glycine-rich protein 2 [Homo sapiens] |
| gi\|4503107 | cystatin C precursor [Homo sapiens] |
| gi\|4503139 | cathepsin B preproprotein [Homo sapiens] |
| gi\|4503211 | cytochrome P450, family 27, subfamily A, polypeptide 1 precursor [Homo sapiens] |
| gi\|18426915 | drebrin 1 isoform a [Homo sapiens] |
| gi\|62530384 | dodecenoyl-Coenzyme A delta isomerase precursor [Homo sapiens] |
| gi\|56682959 | dynactin 1 isoform 1 [Homo sapiens] |
| gi\|4758138 | DEAD (Asp-Glu-Ala-Asp) box polypeptide 5 [Homo sapiens] |
| gi\|193783649 | unnamed protein product [Homo sapiens] |
| gi\|42544174 | deoxythymidylate kinase (thymidylate kinase) [Homo sapiens] |
| gi\|4503471 | eukaryotic translation elongation factor 1 alpha 1 [Homo sapiens] |
| gi\|4503475 | eukaryotic translation elongation factor 1 alpha 2 [Homo sapiens] |
| gi\|4503477 | eukaryotic translation elongation factor 1 beta 2 [Homo sapiens] |
| gi\|4503481 | eukaryotic translation elongation factor 1 gamma [Homo sapiens] |
| gi\|4503483 | eukaryotic translation elongation factor 2 [Homo sapiens] |
| gi\|4826708 | ephrin A3 [Homo sapiens] |
| gi\|110347457 | EGF-like-domain, multiple 3 [Homo sapiens] |
| gi\|4503529 | eukaryotic translation initiation factor 4A isoform 1 [Homo sapiens] |
| gi\|49355765 | ELAV-like protein 3 isoform 2 [Homo sapiens] |
| gi\|4503571 | enolase 1 [Homo sapiens] |
| gi\|4758272 | endosulfine alpha isoform 3 [Homo sapiens] |
| gi\|166362737 | erythrocyte membrane protein band 4.2 isoform 2 [Homo sapiens] |
| gi\|194097332 | ephrin receptor EphB6 precursor [Homo sapiens] |
| gi\|4503599 | excision repair cross-complementing 1 isofrom 2 [Homo sapiens] |
| gi\|46370066 | exostosin 1 [Homo sapiens] |
| gi\|27886588 | PTK2B protein tyrosine kinase 2 beta isoform b [Homo sapiens] |
| gi\|41872631 | fatty acid synthase [Homo sapiens] |
| gi\|4503659 | ubiquitin-like protein fubi and ribosomal protein S30 precursor [Homo sapiens] |
| gi\|67089147 | farnesyl-diphosphate farnesyltransferase 1 [Homo sapiens] |
| gi\|209571584 | farnesyl diphosphate synthase isoform b [Homo sapiens] |
| gi\|4758356 | flap structure-specific endonuclease 1 [Homo sapiens] |
| gi\|213417614 | fibroblast growth factor 13 isoform 3 [Homo sapiens] |
| gi\|4503711 | fibroblast growth factor receptor 3 isoform 1 precursor [Homo sapiens] |
| gi\|4503727 | FK506 binding protein 3, 25 kDa [Homo sapiens] |
| gi\|116829964 | KiSS-1 metastasis-suppressor [Homo sapiens] |
| gi\|194306559 | kinesin light chain 1 isoform 3 [Homo sapiens] |
| gi\|9845502 | ribosomal protein SA [Homo sapiens] |
| gi\|5031851 | stathmin 1 [Homo sapiens] |
| gi\|4557032 | L-lactate dehydrogenase B [Homo sapiens] |
| gi\|5031877 | lamin B1 [Homo sapiens] |
| gi\|167555125 | low density lipoprotein receptor-related protein 3 [Homo sapiens] |
| gi\|153945728 | microtubule-associated protein 1B [Homo sapiens] |
| gi\|8400715 | microtubule-associated protein tau isoform 4 [Homo sapiens] |
| gi\|14043022 | methionyl-tRNA synthetase [Homo sapiens] |
| gi\|110347461 | MYC-associated zinc finger protein isoform 1 [Homo sapiens] |
| gi\|6631095 | minichromosome maintenance complex component 3 [Homo sapiens] |
| gi\|23510448 | minichromosome maintenance complex component 5 [Homo sapiens] |
| gi\|5031905 | MyoD family inhibitor [Homo sapiens] |
| gi\|84311247 | mutant truncated midkine B [Homo sapiens] |
| gi\|57222568 | myeloid/lymphoid or mixed-lineage leukemia (trithorax homolog, Drosophila); |
| | translocated to, 6 [Homo sapiens] |
| gi\|61835204 | mercaptopyruvate sulfurtransferase isoform 2 [Homo sapiens] |
| gi\|4505289 | diphosphomevalonate decarboxylase [Homo sapiens] |
| gi\|17986258 | myosin, light chain 6, alkali, smooth muscle and non-muscle isoform 1 [Homo sapiens] |
| gi\|154354979 | myosin X [Homo sapiens] |
| gi\|5174607 | NGFI-A binding protein 2 [Homo sapiens] |
| gi\|5031931 | nascent polypeptide-associated complex alpha subunit isoform b [Homo sapiens] |
| gi\|157412270 | heterogeneous nuclear ribonucleoprotein M isoform b [Homo sapiens] |
| gi\|4557789 | norrin [Homo sapiens] |
| gi\|4758768 | NADH dehydrogenase (ubiquinone) 1 alpha subcomplex, 10, 42 kDa precursor [Homo |
| | sapiens] |
| gi\|4758774 | NADH dehydrogenase (ubiquinone) 1 beta subcomplex, 10, 22 kDa [Homo sapiens] |
| gi\|20149568 | NADH dehydrogenase (ubiquinone) flavoprotein 1, 51 kDa precursor [Homo sapiens] |
| gi\|98986461 | neurogenic differentiation 2 [Homo sapiens] |
| gi\|187608777 | NF-kappa-B inhibitor-like protein 2 [Homo sapiens] |
| gi\|119596463 | neuronatin, isoform CRA_a [Homo sapiens] |
| gi\|4505409 | non-metastatic cells 2, protein (NM23B) expressed in [Homo sapiens] |
| gi\|10835063 | nucleophosmin 1 isoform 1 [Homo sapiens] |
| gi\|34098946 | nuclease sensitive element binding protein 1 [Homo sapiens] |
| gi\|71361682 | nuclear mitotic apparatus protein 1 [Homo sapiens] |
| gi\|9845505 | ornithine decarboxylase antizyme 1 [Homo sapiens] |
| gi\|38372882 | RecName: Full = Mitochondrial inner membrane protein OXA1L; AltName: |
| | Full = Oxidase assembly 1-like protein; Short = OXA1-like protein; AltName: |
| | Full = OXA1Hs; AltName: Full = Hsa; Flags: Precursor |
| gi\|20070125 | prolyl 4-hydroxylase, beta subunit precursor [Homo sapiens] |
| gi\|124494254 | ErbB3-binding protein 1 [Homo sapiens] |
| gi\|4505621 | prostatic binding protein [Homo sapiens] |
| gi\|4505591 | peroxiredoxin 1 [Homo sapiens] |
| gi\|93141029 | paralemmin isoform 2 [Homo sapiens] |
| gi\|106049292 | pyruvate carboxylase precursor [Homo sapiens] |
| gi\|134019487 | chromatin modifying protein 1A isoform 1 [Homo sapiens] |
| gi\|4505685 | pyruvate dehydrogenase (lipoamide) alpha 1 precursor [Homo sapiens] |
| gi\|22202633 | prefoldin subunit 5 isoform alpha [Homo sapiens] |
| gi\|11321601 | phosphofructokinase, platelet [Homo sapiens] |
| gi\|16753215 | profilin 2 isoform a [Homo sapiens] |
| gi\|4505753 | phosphoglycerate mutase 1 (brain) [Homo sapiens] |
| gi\|20149543 | placental growth factor, vascular endothelial growth factor-related protein [Homo |
| | sapiens] |
| gi\|5453898 | protein (peptidyl-prolyl cis/trans isomerase) NIMA-interacting 1 [Homo sapiens] |
| gi\|205360954 | polycystin 1 isoform 1 precursor [Homo sapiens] |
| gi\|33286420 | pyruvate kinase, muscle isoform M1 [Homo sapiens] |
| gi\|33598948 | phospholipase C gamma 1 isoform a [Homo sapiens] |
| gi\|9945439 | septin 5 [Homo sapiens] |
| gi\|156616275 | polymerase (DNA directed), delta 1, catalytic subunit 125 kDa [Homo sapiens] |
| gi\|110618253 | mitochondrial DNA-directed RNA polymerase precursor [Homo sapiens] |
| gi\|127139033 | cytochrome P450 reductase [Homo sapiens] |
| gi\|10863927 | peptidylprolyl isomerase A [Homo sapiens] |
| gi\|4758950 | peptidylprolyl isomerase B precursor [Homo sapiens] |
| gi\|4826932 | peptidylprolyl isomerase D [Homo sapiens] |
| gi\|4506003 | protein phosphatase 1, catalytic subunit, alpha isoform 1 [Homo sapiens] |
| gi\|21361399 | alpha isoform of regulatory subunit A, protein phosphatase 2 [Homo sapiens] |
| gi\|5453958 | protein phosphatase 5, catalytic subunit [Homo sapiens] |
| gi\|38257139 | protein kinase, cAMP-dependent, regulatory, type I, beta [Homo sapiens] |
| gi\|20128774 | mitogen-activated protein kinase 11 [Homo sapiens] |
| gi\|11386147 | prosaposin isoform a preproprotein [Homo sapiens] |
| gi\|4506193 | proteasome beta 1 subunit [Homo sapiens] |
| gi\|25777602 | proteasome 26S non-ATPase subunit 2 [Homo sapiens] |
| gi\|18543329 | proteasome 26S non-ATPase subunit 9 [Homo sapiens] |
| gi\|4506217 | proteasome 26S non-ATPase subunit 10 isoform 1 [Homo sapiens] |
| gi\|46249388 | phosphoserine phosphatase [Homo sapiens] |
| gi\|28558998 | polypyrimidine tract-binding protein 1 isoform d [Homo sapiens] |
| gi\|4506281 | pleiotrophin [Homo sapiens] |
| gi\|4506367 | RAB3A, member RAS oncogene family [Homo sapiens] |
| gi\|9845511 | ras-related C3 botulinum toxin substrate 1 isoform RacI [Homo sapiens] |
| gi\|4506401 | v-raf-1 murine leukemia viral oncogene homolog 1 [Homo sapiens] |
| gi\|5453555 | ras-related nuclear protein [Homo sapiens] |
| gi\|93277122 | RNA binding motif protein 4 [Homo sapiens] |
| gi\|4506457 | reticulocalbin 2, EF-hand calcium binding domain [Homo sapiens] |
| gi\|5454004 | D4, zinc and double PHD fingers family 2 [Homo sapiens] |
| gi\|168983744 | tripartite motif-containing 27 [Homo sapiens] |
| gi\|156416009 | regulator of G-protein signalling 16 [Homo sapiens] |
| gi\|4506649 | ribosomal protein L3 isoform a [Homo sapiens] |
| gi\|16579885 | ribosomal protein L4 [Homo sapiens] |
| gi\|14591909 | ribosomal protein L5 [Homo sapiens] |
| gi\|16753227 | ribosomal protein L6 [Homo sapiens] |
| gi\|15431301 | ribosomal protein L7 [Homo sapiens] |
| gi\|4506661 | ribosomal protein L7a [Homo sapiens] |
| gi\|4506663 | ribosomal protein L8 [Homo sapiens] |
| gi\|15431303 | ribosomal protein L9 [Homo sapiens] |
| gi\|249371 | laminin receptor homolog [Homo sapiens] |
| gi\|15431290 | ribosomal protein L11 [Homo sapiens] |
| gi\|4506597 | ribosomal protein L12 [Homo sapiens] |
| gi\|15431295 | ribosomal protein L13 [Homo sapiens] |
| gi\|15431293 | ribosomal protein L15 [Homo sapiens] |
| gi\|4506617 | ribosomal protein L17 [Homo sapiens] |
| gi\|4506607 | ribosomal protein L18 [Homo sapiens] |
| gi\|11415026 | ribosomal protein L18a [Homo sapiens] |
| gi\|18104948 | ribosomal protein L21 [Homo sapiens] |
| gi\|4506613 | ribosomal protein L22 proprotein [Homo sapiens] |
| gi\|4506621 | ribosomal protein L26 [Homo sapiens] |
| gi\|4506629 | ribosomal protein L29 [Homo sapiens] |
| gi\|4506633 | ribosomal protein L31 isoform 1 [Homo sapiens] |
| gi\|16117791 | ribosomal protein L35a [Homo sapiens] |
| gi\|4506641 | ribosomal protein L37 [Homo sapiens] |
| gi\|4506643 | ribosomal protein L37a [Homo sapiens] |
| gi\|4506667 | ribosomal protein PO [Homo sapiens] |
| gi\|4506671 | ribosomal protein P2 [Homo sapiens] |
| gi\|15055539 | ribosomal protein S2 [Homo sapiens] |
| gi\|15718687 | ribosomal protein S3 [Homo sapiens] |
| gi\|4506723 | ribosomal protein S3a [Homo sapiens] |
| gi\|4506725 | ribosomal protein S4, X-linked X isoform [Homo sapiens] |
| gi\|4506727 | ribosomal protein S4, Y-linked 1 Y isoform [Homo sapiens] |
| gi\|13904870 | ribosomal protein S5 [Homo sapiens] |
| gi\|17158044 | ribosomal protein S6 [Homo sapiens] |
| gi\|4506743 | ribosomal protein S8 [Homo sapiens] |
| gi\|14141193 | ribosomal protein S9 [Homo sapiens] |
| gi\|4506679 | ribosomal protein S10 [Homo sapiens] |
| gi\|14277700 | ribosomal protein S12 [Homo sapiens] |
| gi\|4506685 | ribosomal protein S13 [Homo sapiens] |
| gi\|5032051 | ribosomal protein S14 [Homo sapiens] |
| gi\|4506687 | ribosomal protein S15 [Homo sapiens] |
| gi\|14165469 | ribosomal protein S15a [Homo sapiens] |
| gi\|4506691 | ribosomal protein S16 [Homo sapiens] |
| gi\|11968182 | ribosomal protein S18 [Homo sapiens] |
| gi\|4506701 | ribosomal protein S23 [Homo sapiens] |
| gi\|214010224 | ribosomal protein S24 isoform f [Homo sapiens] |
| gi\|4506707 | ribosomal protein S25 [Homo sapiens] |
| gi\|4506711 | ribosomal protein S27 [Homo sapiens] |
| gi\|4506713 | ubiquitin and ribosomal protein S27a precursor [Homo sapiens] |
| gi\|71772583 | ribosomal protein S29 isoform 2 [Homo sapiens] |
| gi\|45827776 | reticulon 1 isoform C [Homo sapiens] |
| gi\|16306548 | seryl-tRNA synthetase [Homo sapiens] |
| gi\|666052 | syndecan-1 [Homo sapiens] |
| gi\|119577915 | selenoprotein W, 1 [Homo sapiens] |
| gi\|5902076 | splicing factor, arginine/serine-rich 1 isoform 1 [Homo sapiens] |
| gi\|72534660 | splicing factor, arginine/serine-rich 7 [Homo sapiens] |
| gi\|4506913 | sarcoglycan, beta [Homo sapiens] |
| gi\|21389315 | solute carrier family 25 (mitochondrial carrier; citrate transporter), member 1 precursor |
| | [Homo sapiens] |
| gi\|21071056 | SWI/SNF-related matrix-associated actin-dependent regulator of chromatin a4 |
| | isoform B [Homo sapiens] |
| gi\|55956801 | SWI/SNF related, matrix associated, actin dependent regulator of chromatin, |
| | subfamily b, member 1 isoform b [Homo sapiens] |
| gi\|4507115 | fascin 1 [Homo sapiens] |
| gi\|29568103 | U1 small nuclear ribonucleoprotein 70 kDa [Homo sapiens] |
| gi\|4507125 | small nuclear ribonucleoprotein polypeptide B/B′ isoform B [Homo sapiens] |
| gi\|4507127 | small nuclear ribonucleoprotein polypeptide C [Homo sapiens] |
| gi\|4507171 | secreted protein, acidic, cysteine-rich [Homo sapiens] |
| gi\|5902122 | spectrin, beta, non-erythrocytic 2 [Homo sapiens] |
| gi\|38679884 | sorcin isoform b [Homo sapiens] |
| gi\|63253298 | spermidine synthase [Homo sapiens] |
| gi\|149999611 | signal recognition particle 14 kDa (homologous Alu RNA binding protein) [Homo sapiens] |
| gi\|15208660 | tripartite motif protein 21 [Homo sapiens] |
| gi\|108796056 | TROVE domain family, member 2 isoform 1 [Homo sapiens] |
| gi\|4507239 | signal sequence receptor, beta precursor [Homo sapiens] |
| gi\|94400932 | syntaxin 5 [Homo sapiens] |
| gi\|29540543 | sulfotransferase family, cytosolic, 1A, phenol-preferring, member 1 isoform b [Homo |
| | sapiens] |
| gi\|92859638 | synaptotagmin V [Homo sapiens] |
| gi\|4507373 | beta-tubulin cofactor C [Homo sapiens] |
| gi\|41350333 | beta-tubulin cofactor D [Homo sapiens] |
| gi\|4507385 | transcription elongation factor A protein 2 isoform a [Homo sapiens] |
| gi\|57863259 | T-complex protein 1 isoform b [Homo sapiens] |
| gi\|32189392 | peroxiredoxin 2 isoform a [Homo sapiens] |
| gi\|4507521 | transketolase isoform 1 [Homo sapiens] |
| gi\|21265104 | tetraspanin 7 [Homo sapiens] |
| gi\|4507645 | triosephosphate isomerase 1 [Homo sapiens] |
| gi\|4507677 | heat shock protein 90 kDa beta, member 1 [Homo sapiens] |
| gi\|58761484 | chaperonin containing TCP1, subunit 3 isoform c [Homo sapiens] |
| gi\|91208423 | thyroid receptor-interacting protein 6 [Homo sapiens] |
| gi\|116256350 | tuberous sclerosis 2 isoform 4 [Homo sapiens] |
| gi\|49640009 | tetratricopeptide repeat domain 3 [Homo sapiens] |
| gi\|4507729 | tubulin, beta 2 [Homo sapiens] |
| gi\|5803207 | U2 small nuclear RNA auxiliary factor 1 isoform a [Homo sapiens] |
| gi\|67191208 | ubiquitin C [Homo sapiens] |
| gi\|21361091 | ubiquitin carboxyl-terminal esterase L1 [Homo sapiens] |
| gi\|46593007 | ubiquinol-cytochrome c reductase core protein I [Homo sapiens] |
| gi\|46877105 | upstream stimulatory factor 2 isoform 2 [Homo sapiens] |
| gi\|4507877 | vinculin isoform VCL [Homo sapiens] |
| gi\|6005942 | valosin-containing protein [Homo sapiens] |
| gi\|4507879 | voltage-dependent anion channel 1 [Homo sapiens] |
| gi\|62414289 | vimentin [Homo sapiens] |
| gi\|88853069 | vitronectin precursor [Homo sapiens] |
| gi\|190684675 | X-ray repair cross complementing protein 1 [Homo sapiens] |
| gi\|4507949 | tyrosine 3-monooxygenase/tryptophan 5-monooxygenase activation protein, beta |
| | polypeptide [Homo sapiens] |
| gi\|5803225 | tyrosine 3/tryptophan 5-monooxygenase activation protein, epsilon polypeptide |
| | [Homo sapiens] |
| gi\|4507951 | tyrosine 3-monooxygenase/tryptophan 5-monooxygenase activation protein, eta |
| | polypeptide [Homo sapiens] |
| gi\|4507961 | zinc finger protein 36, C3H type, homolog [Homo sapiens] |
| gi\|4827071 | zinc finger protein 9 isoform 3 [Homo sapiens] |
| gi\|23510455 | zinc finger protein 41 [Homo sapiens] |
| gi\|27545332 | zinc finger protein 133 [Homo sapiens] |
| gi\|145977222 | zinc finger protein 154 [Homo sapiens] |
| gi\|32698678 | zinc finger protein 213 [Homo sapiens] |
| gi\|17986283 | tubulin, alpha 1a [Homo sapiens] |
| gi\|197333755 | interferon-related developmental regulator 2 [Homo sapiens] |
| gi\|54607089 | semaphorin 3B isoform 2 precursor [Homo sapiens] |
| gi\|4758112 | HLA-B associated transcript 1 [Homo sapiens] |
| gi\|4502483 | Ras association (Ra1GDS/AF-6) domain family (N-terminal) member 7 [Homo sapiens] |
| gi\|12962937 | achalasia, adrenocortical insufficiency, alacrimia (Allgrove, triple-A) [Homo sapiens] |
| gi\|134142817 | esl protein isoform lb precursor [Homo sapiens] |
| gi\|4507691 | transformation/transcription domain-associated protein [Homo sapiens] |
| gi\|4507183 | speckle-type POZ protein [Homo sapiens] |
| gi\|7657134 | glyceronephosphate 0-acyltransferase [Homo sapiens] |
| gi\|32307161 | cullin 1 [Homo sapiens] |
| gi\|41872527 | diacylglycerol kinase zeta isoform 3 [Homo sapiens] |
| gi\|32189362 | PTPRF interacting protein alpha 3 [Homo sapiens] |
| gi\|55769587 | probable nucleolar complex protein 14 [Homo sapiens] |
| gi\|117553627 | acetylserotonin 0-methyltransferase-like [Homo sapiens] |
| gi\|189571687 | nuclear autoantigen of 14 kDa [Homo sapiens] |
| gi\|16933557 | dachsous 1 precursor [Homo sapiens] |
| gi\|4505813 | dynein light chain 1 [Homo sapiens] |
| gi\|4503525 | eukaryotic translation initiation factor 3, subunit C [Homo sapiens] |
| gi\|4503523 | eukaryotic translation initiation factor 3 subunit D [Homo sapiens] |
| gi\|4503515 | eukaryotic translation initiation factor 3, subunit 3 gamma, 40 kDa [Homo sapiens] |
| gi\|4507285 | syntaxin 10 [Homo sapiens] |
| gi\|4505705 | phosphoprotein enriched in astrocytes 15 [Homo sapiens] |
| gi\|4506903 | splicing factor, arginine/serine-rich 9 [Homo sapiens] |
| gi\|4504079 | glycosylphosphatidylinositol anchor attachment protein 1 [Homo sapiens] |
| gi\|46909598 | a disintegrin and metalloproteinase domain 15 isoform 5 preproprotein [Homo sapiens] |
| gi\|4505229 | Fas-associated via death domain [Homo sapiens] |
| gi\|47933379 | N-ethylmaleimide-sensitive factor attachment protein, alpha [Homo sapiens] |
| gi\|19923191 | minichromosome maintenance complex component 3 associated protein [Homo |
| | sapiens] |
| gi\|14210504 | adaptor-related protein complex 1, mu 1 subunit isoform 2 [Homo sapiens] |
| gi\|4507913 | Wiskott-Aldrich syndrome protein family member 1 [Homo sapiens] |
| gi\|48762942 | huntingtin interacting protein-I-related [Homo sapiens] |
| gi\|78000181 | ribosomal protein L14 [Homo sapiens] |
| gi\|11141861 | claudin 9 [Homo sapiens] |
| gi\|115527080 | metastasis associated protein [Homo sapiens] |
| gi\|4759276 | RNA, U3 small nucleolar interacting protein 2 [Homo sapiens] |
| gi\|190358517 | RAB11B, member RAS oncogene family [Homo sapiens] |
| gi\|284005309 | ATP-dependent DNA helicase Q4 [Homo sapiens] |
| gi\|3834621 | homer-3 [Homo sapiens] |
| gi\|10834682 | PP3958 [Homo sapiens] |
| gi\|33695117 | p53-induced protein [Homo sapiens] |
| gi\|118344454 | sperm associated antigen 7 [Homo sapiens] |
| gi\|4758496 | H2A histone family, member Y isoform 2 [Homo sapiens] |
| gi\|195927015 | phosphatidylinositol transfer protein, membrane-associated isoform a [Homo sapiens] |
| gi\|116256445 | nuclear receptor co-repressor 2 isoform 2 [Homo sapiens] |
| gi\|22027622 | TNF receptor-associated factor 4 [Homo sapiens] |
| gi\|57242774 | hypothetical protein LOC9703 [Homo sapiens] |
| gi\|7661916 | lysosomal protein transmembrane 4 alpha [Homo sapiens] |
| gi\|7661890 | sorting nexin 17 [Homo sapiens] |
| gi\|17999539 | DEAH (Asp-Glu-Ala-His) box polypeptide 38 [Homo sapiens] |
| gi\|55769518 | inositol hexakisphosphate kinase 1 isoform 2 [Homo sapiens] |
| gi\|110624774 | mannose receptor, C type 2 [Homo sapiens] |
| gi\|55925608 | kelch-like 21 [Homo sapiens] |
| gi\|151108509 | Rho GTPase-activating protein RICH2 [Homo sapiens] |
| gi\|55770886 | ubiquitin specific protease 3 [Homo sapiens] |
| gi\|14110407 | heterogeneous nuclear ribonucleoprotein D-like [Homo sapiens] |
| gi\|62750347 | histone deacetylase 5 isoform 1 [Homo sapiens] |
| gi\|194294519 | nuclear receptor subfamily 1, group H, member 3 isoform c [Homo sapiens] |
| gi\|65301139 | ATPase, class II, type 9A [Homo sapiens] |
| gi\|5031595 | actin related protein 2/3 complex subunit 4 isoform a [Homo sapiens] |
| gi\|6912540 | nucleotide binding protein 2 (MinD homolog, E. coli) [Homo sapiens] |
| gi\|5031569 | ARP1 actin-related protein 1 homolog A, centractin alpha [Homo sapiens] |
| gi\|155722983 | TNF receptor-associated protein 1 [Homo sapiens] |
| gi\|121114294 | RAS p21 protein activator 4 isoform 1 [Homo sapiens] |
| gi\|5031633 | FERM, RhoGEF, and pleckstrin domain protein 1 isoform 1 [Homo sapiens] |
| gi\|5032031 | RNA binding motif protein 5 [Homo sapiens] |
| gi\|5032133 | eukaryotic translation initiation factor 1 [Homo sapiens] |
| gi\|74735668 | RecName: Full = Major facilitator superfamily domain-containing protein 10; |
| | AltName: Full = Tetracycline transporter-like protein |
| gi\|5031669 | cyclin-dependent kinase 2 associated protein 2 [Homo sapiens] |
| gi\|56181387 | STIP1 homology and U-box containing protein 1 [Homo sapiens] |
| gi\|95147542 | amyloid beta precursor protein-binding, family B, member 3 isoform a [Homo sapiens] |
| gi\|116256481 | TNFAIP3 interacting protein 1 [Homo sapiens] |
| gi\|57013276 | tubulin, alpha, ubiquitous [Homo sapiens] |
| gi\|50592996 | tubulin, beta, 4 [Homo sapiens] |
| gi\|21361322 | tubulin, beta 4 [Homo sapiens] |
| gi\|5174735 | tubulin, beta, 2 [Homo sapiens] |
| gi\|37655183 | N-myc downstream regulated 1 [Homo sapiens] |
| gi\|5174447 | guanine nucleotide binding protein (G protein), beta polypeptide 2-like 1 [Homo sapiens] |
| gi\|5454064 | RNA binding motif protein 14 [Homo sapiens] |
| gi\|17136143 | olfactomedin related ER localized protein isoform 1 [Homo sapiens] |
| gi\|29893564 | microspherule protein 1 isoform 1 [Homo sapiens] |
| gi\|5174723 | translocase of outer mitochondrial membrane 40 [Homo sapiens] |
| gi\|9257197 | BAII-associated protein 2 isoform 1 [Homo sapiens] |
| gi\|19923354 | zinc finger protein 238 isoform 2 [Homo sapiens] |
| gi\|23397429 | eukaryotic translation initiation factor 3, subunit M [Homo sapiens] |
| gi\|5453595 | adenylyl cyclase-associated protein [Homo sapiens] |
| gi\|5454166 | vesicle transport through interaction with t-SNAREs 1B [Homo sapiens] |
| gi\|18379349 | vesicle amine transport protein 1 [Homo sapiens] |
| gi\|6102858 | hypothetical protein [Homo sapiens] |
| gi\|36287060 | K(lysine) acetyltransferase 5 isoform 3 [Homo sapiens] |
| gi\|5453838 | Sjogren syndrome/scleroderma autoantigen 1 [Homo sapiens] |
| gi\|95113651 | glutaredoxin 3 [Homo sapiens] |
| gi\|5453629 | dynactin 2 [Homo sapiens] |
| gi\|26051229 | mitochondrial ribosomal protein L28 [Homo sapiens] |
| gi\|58331185 | chaperonin containing TCP1, subunit 7 isoform b [Homo sapiens] |
| gi\|197383077 | PDZ and LIM domain 5 isoform a [Homo sapiens] |
| gi\|32454737 | tripartite motif-containing 3 [Homo sapiens] |
| gi\|171184421 | thioredoxin interacting protein [Homo sapiens] |
| gi\|5454030 | RAS-related on chromosome 22 isoform a [Homo sapiens] |
| gi\|5729794 | CUG triplet repeat, RNA-binding protein 1 isoform 1 [Homo sapiens] |
| gi\|10835246 | Kruppel-like factor 1 (erythroid) [Homo sapiens] |
| gi\|5729999 | Ras-related GTP binding A [Homo sapiens] |
| gi\|5729953 | nuclear distribution gene C homolog [Homo sapiens] |
| gi\|38261971 | cyclic AMP-regulated phosphoprotein, 21 kD isoform 1 [Homo sapiens] |
| gi\|28269672 | serologically defined colon cancer antigen 8 [Homo sapiens] |
| gi\|42544159 | heat shock 105 kD [Homo sapiens] |
| gi\|5729875 | progesterone receptor membrane component 1 [Homo sapiens] |
| gi\|5730015 | RUN domain containing 3A [Homo sapiens] |
| gi\|19387846 | melanoma antigen family D, 2 [Homo sapiens] |
| gi\|5802970 | AFG3 ATPase family gene 3-like 2 [Homo sapiens] |
| gi\|5803023 | lectin, mannose-binding 2 precursor [Homo sapiens] |
| gi\|5803013 | endoplasmic reticulum protein 29 isoform 1 precursor [Homo sapiens] |
| gi\|5870893 | solute carrier family 38, member 3 [Homo sapiens] |
| gi\|166795250 | kinesin family member 2C [Homo sapiens] |
| gi\|33286446 | opioid growth factor receptor [Homo sapiens] |
| gi\|34850061 | superiorcervical ganglia, neural specific 10 [Homo sapiens] |
| gi\|16554627 | WD repeat domain 5 [Homo sapiens] |
| gi\|66472382 | nischarin [Homo sapiens] |
| gi\|6005860 | ribosomal protein L35 [Homo sapiens] |
| gi\|9966764 | lysophospholipase II [Homo sapiens] |
| gi\|45446743 | DEAD box polypeptide 42 protein [Homo sapiens] |
| gi\|32528286 | acyl-CoA thioesterase 7 isoform hBACHd [Homo sapiens] |
| gi\|6005764 | GABA(A) receptor-associated protein [Homo sapiens] |
| gi\|11559929 | coatomer protein complex, subunit gamma 1 [Homo sapiens] |
| gi\|17978512 | poly-U binding splicing factor 60 KDa isoform a [Homo sapiens] |
| gi\|41281557 | latrophilin 1 isoform 2 precursor [Homo sapiens] |
| gi\|41349441 | SEC31 homolog A isoform 2 [Homo sapiens] |
| gi\|57242755 | calsyntenin 1 isoform 2 [Homo sapiens] |
| gi\|20336290 | DEAH (Asp-Glu-Ala-His) box polypeptide 30 isoform 2 [Homo sapiens] |
| gi\|21396480 | RNA binding protein (autoantigenic, hnRNP-associated with lethal yellow) short |
| | isoform [Homo sapiens] |
| gi\|66932902 | SREBF chaperone protein [Homo sapiens] |
| gi\|58761546 | amine oxidase (flavin containing) domain 2 isoform b [Homo sapiens] |
| gi\|42516565 | ubiquitin specific protease 33 isoform 2 [Homo sapiens] |
| gi\|83716024 | kinesin family member 21B [Homo sapiens] |
| gi\|24307983 | FtsJ methyltransferase domain containing 2 [Homo sapiens] |
| gi\|122937251 | jumonji domain containing 3, histone lysine demethylase [Homo sapiens] |
| gi\|155722994 | zinc finger CCCH-type containing 3 [Homo sapiens] |
| gi\|67763814 | death-inducing-protein [Homo sapiens] |
| gi\|112421108 | capicua homolog [Homo sapiens] |
| gi\|154689719 | hypothetical protein LOC23231 [Homo sapiens] |
| gi\|21327667 | block of proliferation 1 [Homo sapiens] |
| gi\|62241044 | exocyst complex component 7 isoform b [Homo sapiens] |
| gi\|82659109 | retinoblastoma-associated factor 600 [Homo sapiens] |
| gi\|21624607 | coactosin-like 1 [Homo sapiens] |
| gi\|18034692 | CDW92 antigen [Homo sapiens] |
| gi\|7657455 | pescadillo homolog 1, containing BRCT domain [Homo sapiens] |
| gi\|45827731 | scribble isoform a [Homo sapiens] |
| gi\|6912458 | calcineurin binding protein 1 [Homo sapiens] |
| gi\|6912674 | SNAP-associated protein [Homo sapiens] |
| gi\|7524354 | dimethylarginine dimethylaminohydrolase 2 [Homo sapiens] |
| gi\|164519084 | RAB GTPase activating protein 1 [Homo sapiens] |
| gi\|112363070 | hsp70-interacting protein [Homo sapiens] |
| gi\|72534684 | phospholipase D family, member 3 [Homo sapiens] |
| gi\|6912490 | transcription factor MAFF [Homo sapiens] |
| gi\|52630440 | FK506-binding protein 8 [Homo sapiens] |
| gi\|7657345 | mitochondrial carrier homolog 1 [Homo sapiens] |
| gi\|160420328 | SHC (Src homology 2 domain containing) transforming protein 2 [Homo sapiens] |
| gi\|194306564 | 6-phosphogluconolactonase [Homo sapiens] |
| gi\|6912238 | peroxiredoxin 5 isoform a precursor [Homo sapiens] |
| gi\|4507975 | zinc finger protein 345 [Homo sapiens] |
| gi\|32698704 | hypothetical protein LOC25851 [Homo sapiens] |
| gi\|17530785 | breast cancer metastasis suppressor 1 isoform 1 [Homo sapiens] |
| gi\|36796743 | methylenetetrahydrofolate dehydrogenase (NADP+ dependent) 1-like [Homo sapiens] |
| gi\|20070260 | cofactor of BRCA1 [Homo sapiens] |
| gi\|195972913 | nasal embryonic LHRH factor isoform d [Homo sapiens] |
| gi\|7661696 | hypothetical protein LOC26017 [Homo sapiens] |
| gi\|5911953 | hypothetical protein [Homo sapiens] |
| gi\|41872443 | TRPC4-associated protein isoform b [Homo sapiens] |
| gi\|31542521 | N-acetyltransferase 9 (GCN5-related, putative) [Homo sapiens] |
| gi\|150170699 | kinesin family member 26A [Homo sapiens] |
| gi\|28416431 | GTPase, IMAP family member 2 [Homo sapiens] |
| gi\|215277017 | protein tyrosine phosphatase, non-receptor type 18 isoform 2 [Homo sapiens] |
| gi\|46367787 | poly(A) binding protein, cytoplasmic 1 [Homo sapiens] |
| gi\|7656952 | calcyclin binding protein isoform 1 [Homo sapiens] |
| gi\|7657599 | sorting nexin 5 [Homo sapiens] |
| gi\|148727368 | bromodomain and PHD finger containing, 3 [Homo sapiens] |
| gi\|7657514 | GTP-binding protein RHO6 [Homo sapiens] |
| gi\|140560994 | growth inhibition and differentiation related protein 86 [Homo sapiens] |
| gi\|166235178 | PDZ and LIM domain protein 3 isoform b [Homo sapiens] |
| gi\|7657196 | zinc finger protein 330 [Homo sapiens] |
| gi\|158256596 | unnamed protein product [Homo sapiens] |
| gi\|167466201 | WAS protein family homolog 1 [Homo sapiens] |
| gi\|58219048 | hairy and enhancer of split 5 [Homo sapiens] |
| gi\|157819143 | hypothetical protein LOC402665 [Homo sapiens] |
| gi\|39992616 | PIM3 protein [Homo sapiens] |
| gi\|169161838 | PREDICTED: similar to hCG2040565 [Homo sapiens] |
| gi\|205830928 | RecName: Full = TLC domain-containing protein 2 |
| gi\|119964728 | D-2-hydroxyglutarate dehydrogenase precursor [Homo sapiens] |
| gi\|124249392 | DNL-type zinc finger [Homo sapiens] |

1-18. (canceled)

19. A method for diagnosing systemic lupus erythematosus, comprising
a) contacting a solid support having applied thereon at least one marker sequence of a cDNA selected from the group SEQ 1-716 and/or SEQ 1a-716a, or a respective protein coding therefor, or a respective partial sequence or fragment thereof with body fluid or tissue extract of a patient, and
b) detecting an interaction of the body fluid or tissue extract with said at least one marker sequence.

20. The method for diagnosing systemic lupus erythematosus according to claim 19, wherein said at least one marker sequence is a protein encoded by a cDNA selected from the group SEQ 1-716 and/or SEQ 1a-716a, or a respective partial sequence or fragment thereof, and
wherein said method further comprises normalizing said at least one marker sequence with autoantibodies from patients who do not have systemic lupus erythematosus.

21. A method for risk stratification or for managing the treatment of a patient with systemic lupus erythematosus, comprising
determining the presence of at least one marker sequence of a cDNA selected from the group SEQ 1-716 and/or SEQ 1a-716, or a respective protein coding therefor, or a respective partial sequence or fragment thereof in body fluid or tissue extract of a patient.

22. The method according to claim 21, wherein the stratification or the treatment management comprises decisions regarding the treatment and therapy of the patient, in particular hospitalization of the patient, use, effect and/or dosage of one or more pharmaceuticals, a therapeutic measure, or monitoring the progression of an illness or treatment, etiology, or classification of a disease, including prognosis.

23. An arrangement of marker sequences comprising at least one marker sequence of a cDNA selected from the group SEQ 1-716 and/or SEQ 1a-716a, or a respective protein coding therefor, wherein SEQ 1-716 and/or SEQ 1a-716a are identified by way of a normalized protein biochip.

24. The arrangement according to claim 23, characterized in that at least 2 to 5 or 10, preferably 30 to 50 marker sequences, or 50 to 100 or more marker sequences are present.

25. The arrangement according to claim 23, characterized in that the marker sequences are present in the form of clones.

26. An assay, protein biochip comprising an arrangement according to claim 23, characterized in that the marker sequences are applied to a solid support.
